(19) United States
(12) Patent Application Publication
(10) Pub. No.: US 2018/0235182 A1
(43) Pub. Date: Aug. 23, 2018

(54) ANIMAL MONITORING DEVICE

(71) Applicant: ALPHA VET TECH HOLDINGS PTY LTD, Bowral (AU)

(72) Inventor: Jeremy BOCKNEK, Wollongong (AU)

(21) Appl. No.: 15/554,606

(22) PCT Filed: Mar. 4, 2016
(86) PCT No.: PCT/AU2016/050153

§ 371 Date: Aug. 30, 2017

(30) Foreign Application Priority Data
Mar. 5, 2015 (AU) 2015900770

(51) Int. Cl. A01K 29/00; A61B 5/00; A61B 5/0205; A61B 5/0402; A61B 5/0476; A61D 17/00

(52) U.S. Cl. CPC A01K 29/005; A61B 5/0006; A61B 5/0205; A61B 5/0402; A61B 5/024; A61B 5/6822; A61B 5/0024; A61D 17/00; A61B 5/0022; A61B 5/0476

(57) ABSTRACT

A method for monitoring an animal, the method including an electronic processing device (121) for wirelessly receiving at least one signal indicative of first and second biological attributes of the animal, wherein the at least one first biological attribute is at least partially sensed using at least one first sensor (111) worn on a neck region of the animal; and, the at least one second biological attribute is at least partially sensed using at least one second sensor (112) worn on a non-neck region of the animal; and, generating at least one indicator at least partially indicative of the first and second biological attributes.

### BACKGROUND OF THE INVENTION

The present invention relates to a device and method for animal monitoring, and in particular for monitoring non-human animals.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

It is known to monitor animals, including non-humans, during surgery or treatment in order to detect changes in vital signs indicative of adverse reactions. However, typically such monitoring involves the use of a number of sensors including numerous leads which can obstruct regions of the animal's body which the veterinarian intends to operate on. In addition, due to both the leads and positioning of the various sensors, it is typically not possible to monitor animals either pre-operatively or in post-operative recovery, when the animal is ambulatory, as the animal may attempt to remove or damage the leads and sensors.

Consequently, in veterinary applications pre and post-operative monitoring is typically performed manually either by examination or viewing the animal. In this regard, in order to continually monitor larger numbers of animals, a large number of veterinarians and/or nurses is required which is time consuming and costly.

### SUMMARY OF THE PRESENT INVENTION

The present invention seeks to ameliorate one or more of the problems associated with the prior art.

In one broad form the present invention seeks to provide a method for monitoring an animal, the method including an electronic processing device for:

a) wirelessly receiving at least one signal indicative of first and second biological attributes of the animal, wherein:
i) the at least one first biological attribute is at least partially sensed using at least one first sensor worn on a neck region of the animal; and,
ii) the at least one second biological attribute is at least partially sensed using at least one second sensor worn on a non-neck region of the animal; and,

b) generating at least one indicator at least partially indicative of the first and second biological attributes.

Typically the method includes displaying a representation of the at least one indicator.

Typically the method includes:
a) generating the at least one signal from second signals received from the second sensor using a receiver provided in proximity to the first sensor and from first signals from the first sensor; and,
b) transmitting the at least one signal from a transmitter provided in proximity to the first sensor.

Typically the method includes, in the electronic processing device:
a) determining attribute values indicative of the first and second biological attributes; and,
b) determining the indicator using the attribute values.

Typically the method includes, in the electronic processing device:
a) comparing the attribute values to at least one reference; and,
b) generating the indicator using a result of the comparison.

Typically the reference is at least one of:
a) derived from a normal population;
b) a predetermined threshold;
c) determined from predetermined values; and,
d) indicative of previously determined attribute values.

Typically the previously determined attribute values are determined prior to the animal undergoing at least one of:
a) surgery; and,
b) treatment.

Typically the animal is a non-human animal.

Typically the animal is ambulatory.

Typically the non-neck region includes any one of:
a) a tail; and,
b) a hind leg.

Typically the at least one second biological attribute includes at least one of:
a) a heart rate; and,
b) an oxygen saturation.

Typically the at least one first biological attribute includes at least one of:
a) electrical activity of a heart of the animal; and,
b) a respiration rate.

Typically the second signal is indicative of an electrocardiograph or electrocardiogram.

Typically the first or second sensor includes at least one of:
a) a photodetector;
b) an induction sensor;
c) an elastomeric sensor;
d) a pressure sensor;
e) a current sensor;
f) a voltage sensor;
g) an impedance sensor;
h) a resistance sensor; and,
i) an accelerometer.

In another broad form the present invention seeks to provide an apparatus for use in monitoring an animal, the apparatus including:
a) at least one first sensor worn on a neck region of the animal for at least partially sensing at least one first biological attribute of the animal;
b) at least one second sensor worn on a non-neck region of the animal for at least partially sensing at least one second biological attribute of the animal; and,
c) a base station including an electronic processing device for:
   i) wirelessly receiving at least one signal indicative of the first and second biological attributes; and,
   ii) generating at least one indicator at least partially indicative of the first and second biological attributes.

Typically the apparatus includes a first and second wearable support being worn by the animal, the first and second wearable supports supporting the first and second sensors respectively.

Typically the first wearable support includes a wireless transmitter for transmitting the at least one signal.

Typically the first or second sensor includes at least one of:
a) a photodetector;
b) an induction sensor;
c) an elastomeric sensor;
d) a pressure sensor;
e) a current sensor;
f) a voltage sensor;
g) an impedance sensor;
h) a resistance sensor; and,
i) an accelerometer.

In another broad form the present invention seeks to provide an apparatus for use in monitoring an animal, the apparatus including:
a) a first wearable support being worn at least partially on a neck region of the animal, the first wearable support including:
   i) at least one first sensor for at least partially sensing at least one first biological attribute of the animal and generating a first signal indicative of the at least one first biological attribute;
   ii) a first wireless receiver for receiving at least one second signal indicative of at least one second biological attribute of the animal; and,
   iii) a first wireless transmitter for transmitting at least one signal indicative of the first and second biological attributes using the first and second signals; and,
b) a second wearable support being worn on a non-neck region of the animal, the second wearable support including:
   i) at least one second sensor for at least partially sensing the second biological attribute and generating the second signal; and,
   ii) a second wireless transmitter for transmitting the second signal.

In another broad form the present invention seeks to provide an apparatus that communicates with a collar for monitoring an animal, the apparatus including:
a) a wearable support including at least one sensor for at least partially sensing at least one biological attribute of the animal, the wearable support being worn at least partially on a non-neck region of the animal; and,
b) a wireless transmitter provided on the wearable support for transmitting at least one signal indicative of the biological attribute to the collar.

In another broad form the present invention seeks to provide a collar for use in monitoring an animal, the collar being a wearable support worn at least partially on a neck region of the animal, the collar including:
i) at least one sensor for at least partially sensing at least one first biological attribute of the animal and generating a first signal indicative of the at least one first biological attribute;
ii) a first wireless receiver for receiving at least one second signal indicative of at least one second biological attribute of the animal; and,
iii) a first wireless transmitter for transmitting at least one signal indicative of the first and second biological attributes using the first and second signals.

### BRIEF DESCRIPTION OF THE DRAWINGS

An example of the present invention will now be described with reference to the accompanying drawings, in which:—

FIG. 1A is a schematic diagram of a first example of an apparatus for use in monitoring an animal;

FIG. 1B is a flowchart of a first example of a method for use in monitoring an animal using the apparatus of FIG. 1A;

FIG. 2A is a schematic diagram of a second example of an apparatus for use in monitoring an animal;

FIG. 2B is a schematic diagram of an example of the apparatus of FIG. 2A in use;

FIG. 3 is a flowchart of a second example of a method for use in monitoring an animal;

FIG. 4 is a schematic diagram of a third example of an apparatus for use in monitoring an animal;

FIG. 5 is a flowchart of a third example of a method for use in monitoring an animal;

FIG. 6 is a flowchart of a fourth example of a method for use in monitoring an animal during surgery;

FIGS. 7A to 7C are schematic diagrams of examples of representations indicative of first and second signals;

FIG. 8 is a circuit diagram of an example of a collar for use in monitoring an animal;

FIG. 9 is a circuit diagram of an example of a tail piece for use in monitoring an animal;

FIG. 10A is a schematic diagram of an example of a user interface displayed by an apparatus for use with an apparatus for use in monitoring an animal;

FIG. 10B is a schematic diagram of a further example of a user interface displayed by the apparatus of FIG. 10A;

FIG. 10C is a schematic diagram of a further example of a user interface displayed by the apparatus of FIG. 10A;

FIG. 11A is a schematic diagram of a further example of a tail piece for use in monitoring an animal;

FIG. 11B is a schematic diagram of a further example of a collar for use in monitoring an animal;

FIG. 11C is a schematic diagram of a further example of an apparatus for use in monitoring an animal; and,

FIG. 12 is an image of a further example of an apparatus for use in monitoring an animal.

### DETAILED DESCRIPTION OF THE PREFERRED EMBODIMENTS

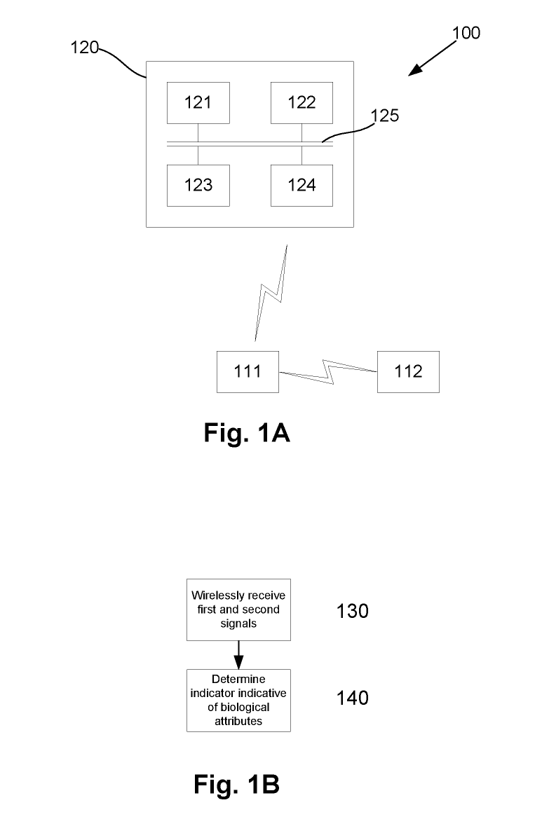

An example of a method and apparatus for monitoring an animal will now be described with reference to FIGS. 1A and 1B.

In this example, the apparatus 100 includes one or more first sensors 111, worn on a neck region of the animal, for at least partially sensing one or more first biological attributes of the animal, and one or more second sensors 112, worn on a non-neck region of the animal, for at least partially sensing one or more second biological attributes of the animal. The apparatus 100 further includes a base station 120 including an electronic processing device 121.

In use, as shown in FIG. 1B, at step 130 the electronic processing device 121 wirelessly receives one or more signals indicative of the first and second biological attribute(s) of the animal. At step 140, the electronic processing device 121 generates at least one indicator at least partially indicative of the first and second biological attributes. A representation of the indicator can then be displayed to an individual, such as a veterinarian, allowing the veterinarian to monitor the physical status of the animal via the electronic processing device. Additionally and/or alternatively, the indictor can be recorded allowing this to act as part of a clinical history of the animal, or presented audibly in the form of an alarm, or the like.

Accordingly, the above described arrangement allows at least two biological attributes of the animal to be sensed via separate sensors worn on different parts of an animal. Signals indicative of the biological attributes are wirelessly received by the electronic processing device and then used to generate one or more indicators indicative of the biological attributes. These indicators can then be used in monitoring animals, for example, during or following surgery, during training or the like. This is becoming more important as types of surgery are expanding and as surgery becomes more complex, for example in organ transplant scenarios or the like.

This arrangement offers a number of advantages.

In particular, as the first and second sensors 111, 112 are worn by the animal, this allows the animal to move, largely unconstrained, which is in contrast to existing sensors that must be clipped, or adhered to the animal and connected to a monitoring apparatus via one or more leads. The combination of wearable sensors and a wireless receiver also allows the animal to be monitored while ambulatory, and thus may be utilised to monitor an animal when not sedated or anesthetised, and optionally continuously and/or over long periods of time.

Furthermore, this arrangement is less likely to cause the animal stress or irritation and may be positioned such that the animal may wear the first and second sensors with their movement and activity unimpeded by large, heavy or awkwardly attached sensors and/or leads/wires, and such that the animal cannot remove or damage the sensors 111, 112. In one example, the first sensor 111 may be worn on the neck as a collar, and the second sensor 112 may be worn on the tail or a hind leg.

The above described arrangement also allows biological attributes to be sensed at multiple regions on an animal, including a neck region and any other non-neck region. In some examples, the non-neck region includes a tail, or hind leg, and in this regard allows improved monitoring of biological attributes such as blood oxygenation, respiratory rate, and heart rate. However, this is not essential and the non-neck region may include any other suitable region for sensing the desired second biological attribute.

Additionally, the arrangement described above provide numerous advantages in a range of industries including veterinary, zoology, farming, and the horse and zoo keeping industries, and in this regard, a wide range of animals including dogs, horses, cattle, cats, birds, reptiles, and the like, may be monitored. In this regard, any type of animal may be monitored, and in one example the animal includes a non-human animal.

In one example, the arrangement may be used to monitor animals during one or more of pre-operative care, surgery, post-operative care, and in addition for long term monitoring, routine examinations, during transport/training, and the like. During pre-operative care the described arrangement allows monitoring of an animal in order to assess their suitability for surgery, such as whether they have an arrhythmia, or other condition, which may increase risks associated with surgery. During surgery, the lack of leads also allows the surgeon to conduct the operation or treatment unobstructed.

Other advantages include that fewer people are required to monitor larger numbers of animals, as multiple first and second sensors can be arranged to communicate with a single base station, thus reducing manpower and hence costs. In addition, animals may be monitored remotely thus enabling more intensive long term monitoring which can aid in identifying and treating conditions earlier.

In other industries, such as the horse industry, the apparatus and method may be utilised in monitoring expensive or rare animals, such as thoroughbreds, to ensure any conditions are identified early and/or pre-empted. For example, foaling mares can be remotely monitored, thus saving time and money, and in addition any conditions which the mare may develop during labour can be identified early and treated. During pregnancy, monitoring can aid in identifying and treating any conditions which may lead to miscarriage, birth abnormalities, or the like, thus enhancing breeding timelines and reducing costs. Further, colts and phillies may be monitored after birth in order to reduce potential risks and mortality rates. Also, stallions may be monitored for optimal collection times, for example, based on the stallion's stress levels.

Additionally, the apparatus and/or method can be utilised during training, for example in training thoroughbred horses, in order to maximise training regimes and/or identify and treat any conditions which may affect performance. Furthermore, monitoring animals during transport can aid in ensuring an animal is not stressed and/or taking pre-emptive action and/or in order to motivate pre-race training regimes. Insurance risks for maintaining and shipping animals may also be reduced with continuous monitoring of the animals. Thus, the system could be used to allow insurance to be provided when this would not otherwise be the case, or to lower premiums, by reducing the chance of adverse events.

The apparatus and/or method also offers a number of advantages in the zoo keeping industry. For example, dangerous and/or wide-roaming animals may be safely and continuously monitored, either for any one or more of the medical purposes outlined above, or for research. For example, it may be desirable to monitor biological attributes of an animal when initially introduced to a new environment, when co-habiting with a potential mate, during infancy, during transport, or to continuously monitor stress levels.

A number of further features will now be described.

Whilst FIG. 1A shows one first sensor and one second sensor, any number of first and second sensors may be used. In addition, the nature of the first and second sensors will vary depending on the preferred implementation, and can include any sensors capable of sensing first and second biological attributes, respectively. For example, the first and/or second sensors 111, 112 can include any one or more of a photodetector, an inductance sensor, an elastomeric sensor, a pressure sensor, a current or a voltage sensor, an impedance or a resistance sensor, an accelerometer, or the like, although any suitable sensor arrangement can also be used.

In this respect, any type of first and second biological attribute may be sensed. In one example, the first and second biological attributes are the same, however this is not essential and in other examples the first and second biological attributes are different. Typically, the first biological attribute includes electrical activity of a heart and/or a respiration rate of the animal, however in other examples may include any one or more of electrical activity along the scalp, a heart rate, and oxygen saturation. Typically, the second biological attribute includes any one or more of a heart rate, and oxygen saturation, however in other examples may additionally or alternatively include electrical activity of a heart of the animal and/or electrical activity along the scalp of the animal and/or a respiration rate. In addition, the first and/or second signal may be indicative of an electrocardiograph or electrocardiogram (ECG or EKG), electroencephalography (EEG), pulse oximetry, heart beats per minute, or the like.

The first and second sensors may be worn in any suitable manner, and in one example first and second wearable supports worn by the animal support the first and second sensors 111, 112, respectively. In this respect, the first and second wearable supports may include any one or more of a collar, a tail piece, a cuff, a strap, tape such as vet-wrap, an adhesive, or the like.

In the example shown in FIG. 1A, the electronic processing device 121 forms part of a processing system 120 including the electronic processing device 121, such as a microprocessor, a memory 122, input/output (I/O) device 123, such as a keyboard and display, and one or more interfaces 124, interconnected via a bus 125. The interfaces 124 may be of any form and can include a Universal Serial Bus (USB) port, Ethernet port, wireless transmitter, or the like, including at least a wireless receiver for coupling to one or more of the first and second sensors 111, 112. In use, the processor 121 receives the one or more first and second signals via the interface 124, optionally storing these in the memory 122. The processor 121 then processes the signals in accordance with instructions stored in the memory 122, for example in the form of software instructions and/or in accordance with input commands provided by a user via the I/O device 123, thereby generating the indicator. The indicator can then be provided as an output, for example via the I/O device 123, or via the interface 124 to a remote processing device, as will be discussed further below.

However, this is for the purpose of example only, and it will be appreciated that the electronic processing device 121 can include any form of electronic processing device that can receive and process first and second signals from the first and second sensors 111, 112. Accordingly, the electronic processing device can include any one or more of a microprocessor, microchip processor, logic gate configuration, firmware optionally associated with implementing logic such as an FPGA (Field Programmable Gate Array), a suitably configured computer system, tablet, smartphone, or any other electronic device, system or arrangement capable of receiving and processing the first and second signals.

The indicator(s) can be of any appropriate form, such as a numerical value representative of values of the biological attributes, or alternatively the result of comparison of biological attribute values to a threshold, as will be described in more detail below.

The indicator(s) can be displayed to a user as part of a representation, such as a graphical, numerical or symbolic representation, as will be discussed in more detail below. Furthermore, the representation of the indicator(s) may be displayed on the display at any suitable time. In one example, the representation is displayed substantially in real-time, and this arrangement may be particularly beneficial for situations such as pre, post and during surgery. In a further example, the indicator(s) may be stored in a store, such as memory 122, and displayed at a later time, for example, when utilised for research applications where real-time monitoring is not required.

In one example, the apparatus 100 includes a first wearable support being worn at least partially on a neck region of the animal, for example in the form of a collar or the like. The first wearable support includes one or more first sensors 111 for at least partially sensing at least one first biological attribute of the animal and generating a first signal indicative of the first biological attribute. The first wearable support also includes a first wireless receiver for receiving at least one second signal indicative of at least one second biological attribute of the animal, and a first wireless transmitter for transmitting at least one signal indicative of the first and second biological attributes using the first and second signals. In this regard, the at least one signal can be a combined signal based on the first and second signals, or could include the first and second signals, sent separately. The first wireless receiver and first wireless transmitter may be provided as separate components, or a single first wireless transceiver.

The apparatus 100 may also include a second wearable support being worn on a non-neck region of the animal, where the second wearable support includes at least one second sensor 112 for at least partially sensing the second biological attribute and, a second wireless transmitter for transmitting the second signal indicative of the second biological attribute.

In one example, the method may include generating the signal from the second signals received using the first wireless receiver and, first signals sensed by the first sensor. Thus, first and second signals indicative of the first and second biological attributes can be combined into a single signal, for example by multiplexing or the like, or alternatively separate first and second signals can be transmitted to the electronic processing device 121. Thus, the first wearable support may include a wireless transmitter for transmitting the signals. However, this is not essential and any suitable arrangement may be used, such as, the first and second signals may be directly received at the base station from first and second transmitters provided in proximity to the first and second sensors, respectively. Alternatively the signal may be generated from first signals received using a receiver provided in proximity to the second sensor 112 and, the first and second signals may be transmitted from a transmitter provided in proximity to the second sensor 112.

As previously described, the electronic processing device 121 typically generates at least one indicator indicative of the first and second biological attributes.

This may be achieved in any suitable manner, and in one example includes determining attribute values indicative of the first and second biological attributes using the at least one signal, and determining the indicator using the attribute values. In one particular example, this includes comparing the attribute values to a reference and, determining the indicator using the results of the comparison. In this instance, the indicator can also be indicative of presence, absence, degree or progression of a condition. In this respect, the reference may be any suitable reference and can be derived from a normal population, a predetermined threshold, and determined from predetermined values. In one example, the reference is indicative of previously determined attribute values, and in this respect the previously determined attribute values may be determined at any suitable time, such as prior to the animal undergoing surgery and/or treatment. This will be discussed in more detail below.

The first and second wearable supports may be provided in a kit, which optionally includes a base station 120, although this is not essential and the first and second wearable supports may be supplied separately.

In this respect, when supplied separately, the second wearable support would correspond to an apparatus that communicates with a collar for monitoring an animal.

Wider variations of the above arrangement are also possible, including incorporating further sensors into the apparatus, such as cameras, motion detectors, global positioning sensors (GPS), laser sensors, and the like. In this regard, the further sensors may transmit signals to the first wearable support, or alternatively directly to the base station. This is particularly useful in a zoo keeping environment, or for research applications, where it may be desirable to monitor the location of animals, their position relative to other animals, and the like.

Further second sensors on non-neck regions may be included in the apparatus such that a single first sensor and multiple second sensors are provided. In this regard, the first wearable support receives multiple second signals indicative of biological attributes from each of the second sensors, and transmits the multiple second signals and first signals sensed at the first sensor, to the base station. The second sensors may sense the same or different biological attributes, and may be placed in similar or different non-neck regions. In one example, the first wearable support may receive second signals from six different second sensors worn on non-neck region(s) of the animal. In this regard, each second sensor may be supported by the same or different second wearable supports. However this is not essential and any suitable number of second sensors may be used.

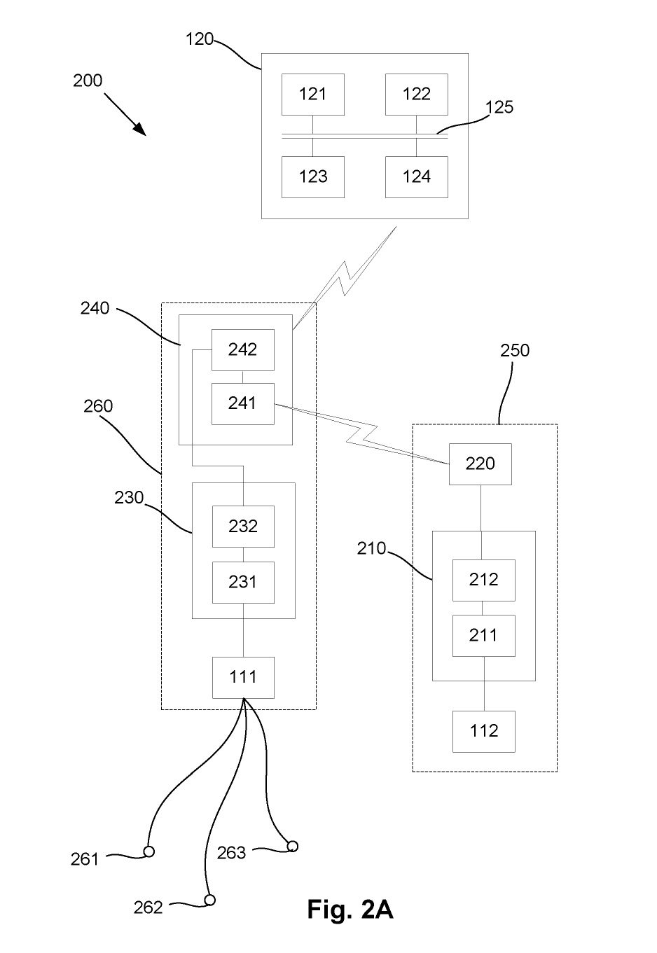

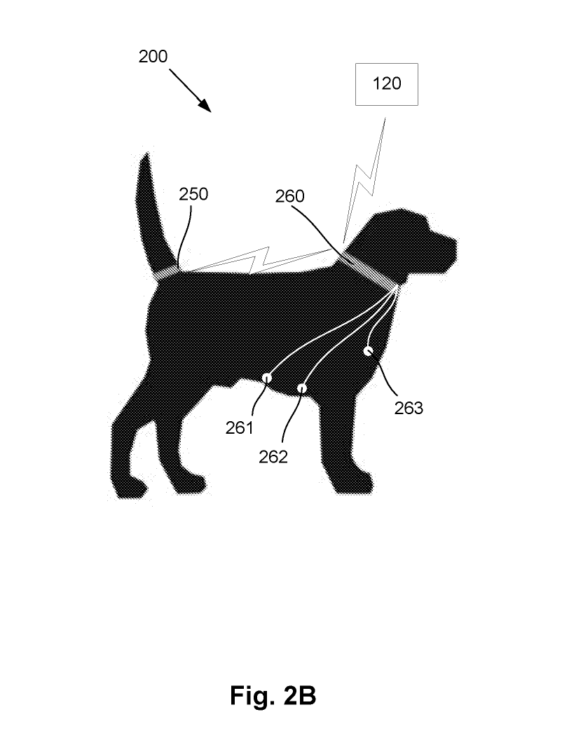

A second example of an apparatus for use in monitoring an animal is shown in FIGS. 2A and 2B. Features similar to those of the example described above have been assigned correspondingly similar reference numerals.

In this example, FIG. 2A provides a schematic diagram of the components of the apparatus 200, including a collar 260, tail piece 250 and base station 120, whilst FIG. 2B provides a schematic diagram of the apparatus 200 of FIG. 2A whilst in use on a dog.

In particular, the collar 260, which corresponds to a first wearable support, includes a first sensor 111, as described above, and a first wireless receiver 241 for receiving second signals indicative of second biological attributes, and a first wireless transmitter 242 for transmitting signals indicative of the first and second biological attributes. The tail piece 250, which corresponds to a second wearable support, is worn on a tail of the animal, typically at the base of the tail, although alternatively could be worn on a hind leg of the animal. The tail piece 250 includes second sensors 112, as described above, and a second wireless transmitter 220 for transmitting the second signals.

The collar 260 may further include an ADC (Analogue to Digital Converter) 232, for sampling analogue signals indicative of the first biological attribute to thereby generate sampled signal values, as well a filter 231 for filtering the analogue signals and/or an amplifier. This can be performed to remove unwanted artefacts, such as noise interference from remote equipment, noise generated by the sensors, as well as to prevent aliasing due to the sampling rate of the ADC, or the like. It will be appreciated that filtering and digitising can be performed in any order, so that filtering can be performed on either or both of the analogue or digitised signals. Similarly, the tail piece 250 may also include an ADC 212 and filter 211 as described above.

Other optional elements which are not shown may include one or more buffers or stores, for temporarily storing at least part of the first and/or second signals, controllers or microprocessors for controlling one of more of signal acquisition, storage, receipt, transmission, or processing. In this respect, the controllers may optionally perform addition signal processing on the digitised signals, such as compression, parameterisation, reconstruction, further filtering, and the like. Indeed, the controllers may incorporate ADC and filtering functionality such that separate ADCs 232, 212 and filters 231, 211, are not required.

Furthermore, the collar 260 and/or tail piece 250 typically include respective power supplies, such as batteries. In this respect, the storage capacity of the batteries, and the wireless protocol, wireless range, and/or other power requirements, will typically effect the battery life. In one example, the collar 260 is capable of transmitting signals up to 1 kilometre and the battery life is approximately 1 hour, however in another example the collar 260 is capable of transmitting signals up to a range of 100 metres and the corresponding battery life is approximately between 4 and 6 hours. However, this is not essential.

In one example, the apparatus 200 may include an intermediary node for amplifying signals being transmitted between the collar 260 and the base station 120, also referred to as signal boosting. In this regard, the intermediary node will typically be positioned remotely from the animal, to receive, amplify and transmit signals being communicated between the base station 120 and collar 260, thereby increasing the potential distance the animal may travel from the base station 120 whilst maintaining communication. In this regard, the intermediary node may include any appropriate power supply, such as a battery or mains power, however more typically the intermediary node will be solar powered. It will be appreciated that this arrangement is particularly useful where the animal is allowed to travel longer distances from the base station 120, such as on a farm, in a zoo, or the like.

In any event, this arrangement offers a number of significant benefits. For example, second signals indicative of the second biological attribute need only be transmitted to the first wireless receiver on the collar 260. In this respect, the wireless protocol of the second wireless transmitter need only be powerful enough to transmit in proximity to the collar 260, and thus may have low power consumption. In a preferred embodiment, the wireless protocol of the second transmitter includes Bluetooth, however this is not essential and in other embodiments may include any one or more of wireless, Zigbee, radio frequency, mobile network, and the like.

In respect of the collar 260, typically the first wireless transmitter requires a longer range than the second wireless transmitter, in order to communicate with the base station 120. In this regard, the wireless protocol of the first wireless transmitter typically includes radio frequency transmission, and in one example using modules sold under the trade name XBEE®, however any suitable wireless protocol may be used including any one or more of Bluetooth, wireless, Zigbee, mobile network, WiFi, or the like. In addition, the first wireless transmitter and first wireless receiver may be provided as separate components, or as a single, first wireless transceiver.

When multiple first and second sensors 111, 112 are available for monitoring multiple animals, is may be desirable to ensure that the base station 120 recognises the particular first and second sensors 111, 112 that are worn by the same animal. In this regard, the collar 260 and/or tail piece 250 may include an input, such as a button, key, or the like, which when activated causes an indicator, such as a light, audible tone, or the like, on the respective tail piece 250/collar 260 to activate. This can be achieved using any of the abovementioned wireless protocols, and in one example using Bluetooth. Thus, a user/operator is able to determine which respective collar 260 and tail piece 250, and hence which first and second sensors 111, 112, are for use on the same animal.

Additionally or alternatively, the collar 260 and tail piece 250 may provide functionality to allow them to be ‘paired’. In this respect, ‘paired’ refers to associating a predetermined collar 260 and tail piece 250 such that the second signal that originates from the tail piece 250 on an animal is received at the collar 260 on the same animal, and hence that the first and second signals received at the base station 120 originate from the same animal.

Therefore, the collar 260 and tail piece 250 may include a request input, such as a button, small interface, or the like, which when activated broadcasts a request for pairing. In this regard, the tail piece or collar 260, respectively, may include an acceptance input, such as a button, key, small interface, or the like, which when subsequently activated accepts the request for pairing, and thus the respective collar 260 and tail piece 250 are ‘paired’.

This ability to detect and/or pair respective collars 260 and tail pieces 250 offers significant benefits, for example, when monitoring multiple animals. In this regard, a user/operator is able to easily identify and/or create paired collars 260 and tail pieces 250, and thus the base station 121 can correctly monitor multiple first and second signals from multiple animals, where each respective first and second signal is indicative of the first and second biological attributes of the same animal.

The collar 260 may include one or more electrodes 261, 262, 263, for positioning on the animal for sensing the first biological attribute. In this example, the electrodes 261, 262, 263 are ECG electrodes for use in sensing electrical activity of the animal's heart, however this is not essential and in other examples the electrodes may include EEG electrodes, for use in sensing electrical activity along the animal's scalp, or the like. The electrodes 261, 262, 263 may be wired to the collar 260, and positioned in any suitable location on the animal, for example, the chest, neck, or the like. In addition, the electrodes 261, 262, 263 may include clips, adhesives, tape or similar in order to secure them on the animal. Furthermore, the tail piece 250 may optionally include electrodes, however this is not essential.

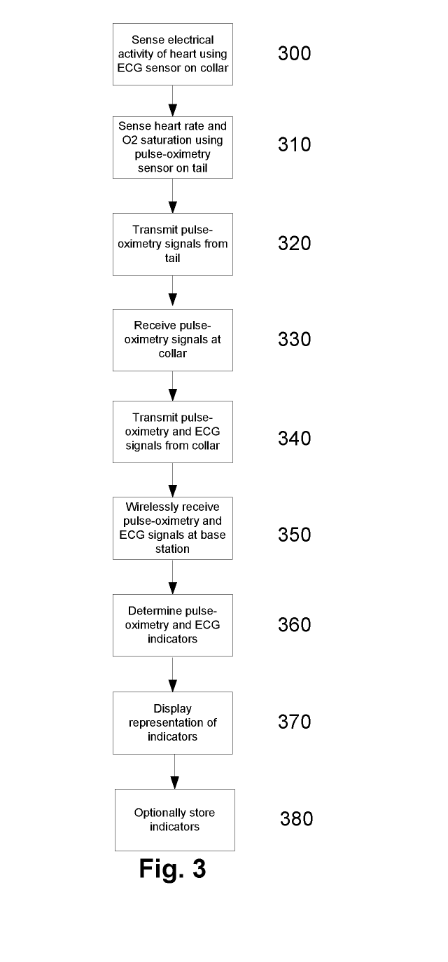

A second example of a method for use in monitoring an animal is shown in FIG. 3.

In this example, at step 300 the method includes sensing electrical activity of the heart of an animal, which corresponds to a first biological attribute, using ECG sensors in communication with the collar 260 worn by the animal.

At step 310 the method includes sensing heart rate and oxygen saturation, which correspond to second biological attributes, of an animal using a pulse-oximetry sensor, such as a photodetector, worn on the tail/hind leg of the animal and supported by the tail piece 250.

At step 320, the pulse-oximetry signals, indicative of the heart rate and oxygen saturation, are transmitted from the tail piece 250, with these being received by the collar 260 at step 330. In this example, the pulse-oximetry signals correspond to the second signals, however as discussed above this is not essential and in other examples the second signals may be generated by further processing the pulse-oximetry signals. Subsequently, the pulse-oximetry signals are received by a receiver on the collar.

In this example, at step 340 the pulse-oximetry and ECG signals are wirelessly transmitted from the collar 260, with these being received by the base station 120 at step 350. In this example, the pulse-oximetry and ECG signals are transmitted sequentially, or by multiplexing the signals, and thus the pulse-oximetry signals correspond to the second signals and the ECG signals correspond to the first signals. Thus, it should be noted that the second signals in this example also correspond to the second signals, however this is not essential. Alternatively, the pulse-oximetry and ECG signals may be further processed before transmission, as discussed above, and in such case the second signals would be indicative of, but not necessarily correspond to, the second signals.

In some examples, a single base station may receive pulse-oximetry and ECG signals from multiple animals, however this is not essential. The base station may subsequently process the pulse-oximetry and/or ECG signals, for example by de-multiplexing, de-compressing, reconstructing, or otherwise processing, the signals.

In addition, transmission of the abovementioned signals may be achieved using any suitable protocol, such as those described above. In one example, transmission may be optionally achieved using a wired connection. For example, during surgery it may be preferable to provide a wired connection between the tail piece and collar and/or collar and base station. In the latter arrangement, legacy and/or existing monitors may be utilised, for example during surgery, thereby negating the need to update all monitors, and thus reducing cost. In addition, wireless transmission may also be provided in conjunction with wired transmission.

At step 360, the method includes generating pulse-oximetry and ECG indicators, which are then displayed on a display at step 370. This may also be achieved in any suitable manner, and in one example the base station includes the display, and the representation includes a graphical and numerical representation of the pulse-oximetry and ECG indicators, such as a graphical trace of the ECG, and numerical values for the number of heart beats per minute and oxygen saturation. However, this is not essential and in other examples the display may be remote from the base station, for example, for remotely displaying the representation on a display over a network, and this will be discussed further below.

Optionally at step 380, the indicators, and/or a representation thereof, may be stored in a store, for example for subsequent review and/or analysis. This can be used for a number of reasons, such as to perform ongoing monitoring of animals in a variety of situations. For example, this could be used to monitor the impact of training and/or transport on performance animals, allowing trainers to optimise pre-race transport and training regimes, as well as during pregnancy to ascertain the likely impact of the current health status of an animal on any offspring. Stored indicators can be used in a data logging process, with stored indicators being time stamped, and optionally encrypted, to prevent subsequent alteration, allowing these to be used in reporting or the like, as will be described in more detail below.

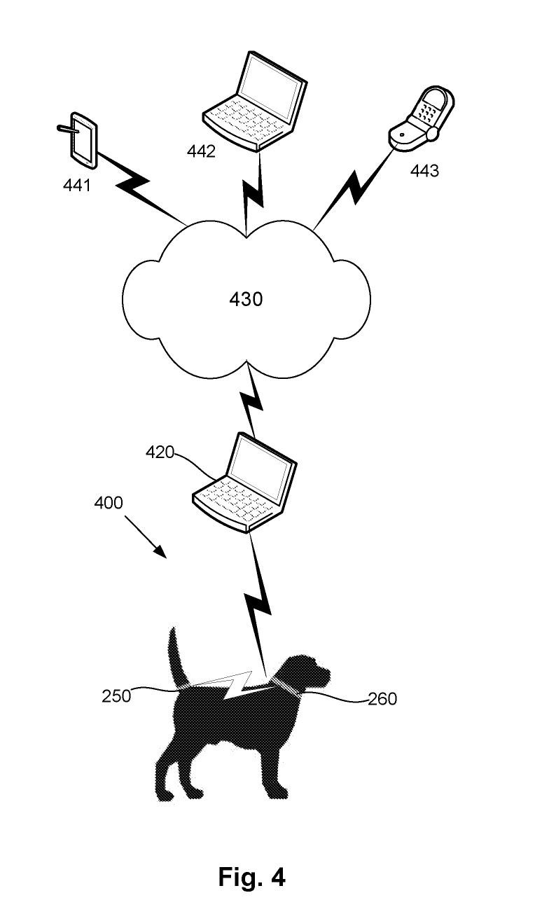

A third example of an apparatus for use in monitoring an animal is shown in FIG. 4. Features similar to those of the example described above have been assigned correspondingly similar reference numerals.

In this example, the apparatus 400 includes a collar 260 and tail piece 250, however any suitable first and second sensor may be used as described above. As these components are described above, they will not be discussed further here.

In addition, the apparatus includes a base station 420 for wirelessly receiving signals indicative of the first and second biological attributes of the animal. In addition, in this example the base station 420 communicates with one or more remote processing systems 441, 442, 443 via a network 430. For example, the remote processing systems 441, 442, 443 may be a remote server, cloud-based application, remote client, or the like for performing specific calculations and/or for remotely displaying a representation indicative of the first and second biological attributes on a remote display. In this respect, signals may be transmitted over the network 430 using any suitable method, such as using the Internet, USB, Ethernet, wireless, XBee®, Bluetooth, mobile network, or the like.

Thus, this arrangement offers a number of advantages, include remote monitoring of animals, which in turn can save on on-site labour, such a veterinarians, researchers, trainers, and the like, and thus save costs. In addition, it allows monitoring of animals in their habitat while not under the influence of a direct observer.

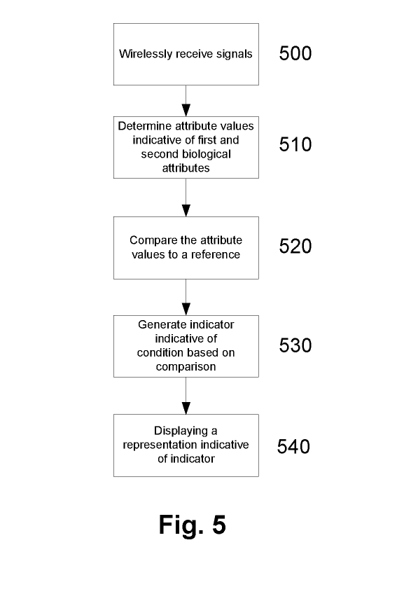

A third example of a method for use in monitoring an animal is shown in FIG. 5.

At step 500 the base station 420 wirelessly receives signals indicative of first and second biological attributes of the animal, respectively. This is achieved according to any one of the arrangements described above.

At step 510, the method includes determining the attribute values indicative of the first and second biological attributes. The attribute values may be determined in any suitable manner and in one example are generated using the signals. For example, the attribute values may be generated by de-multiplexing and/or de-compressing the signals, or optionally via further processing of the signals such as parameterisation, or the like.

At step 520, the method includes comparing the attribute values to a reference. This may be achieved in any suitable manner and in one example the reference is derived from a normal population, a predetermined threshold and/or determined from predetermined values, for example, that are determined prior to the animal undergoing surgery and/or treatment. In a further example, the reference may be indicative of one or more previously determined attribute values. Furthermore, references may be determined based upon breed, gender, species, or the like.

At step 530, the method includes generating an indicator indicative of the presence, absence and/or degree of a condition based upon the results of the comparison. This may be achieved in any suitable manner and in one example includes comparing the results of the comparison to a threshold, or alternatively by classifying the results of the comparison according to a predetermined ranges, or the like.

At step 540, the method further includes displaying and/or otherwise providing a representation indicative of the indicator. In one example, the indicator includes an alert or an alarm, such as any one or more of a sound, a visual indicator, a remote message such as an SMS, email, automated phone call, or the like.

Thus, for example, the base station 420 can monitor the pulse-ox, heart rate or other biological attributes and compare these to predetermined threshold readings. In the event that one of the thresholds is exceeded, then an alarm or other indication can be generated, alerting the veterinary that there is an issue with the health status of the animal.

Wider variations of the above-mentioned arrangement are possible. In one example, the reference, threshold, alert/alarms, and the like may be customisable by a user and/or operator. For example, the user/operator may customise an alarm to indicate when an animal's oxygen saturation falls below a predetermined value. It will be appreciated that customisation allows an operator/user to tailor monitoring for individual animals, for example, in order to receive alerts when slight changes in first and second biological attributes are detected for an animal which is under critical or intensive care.

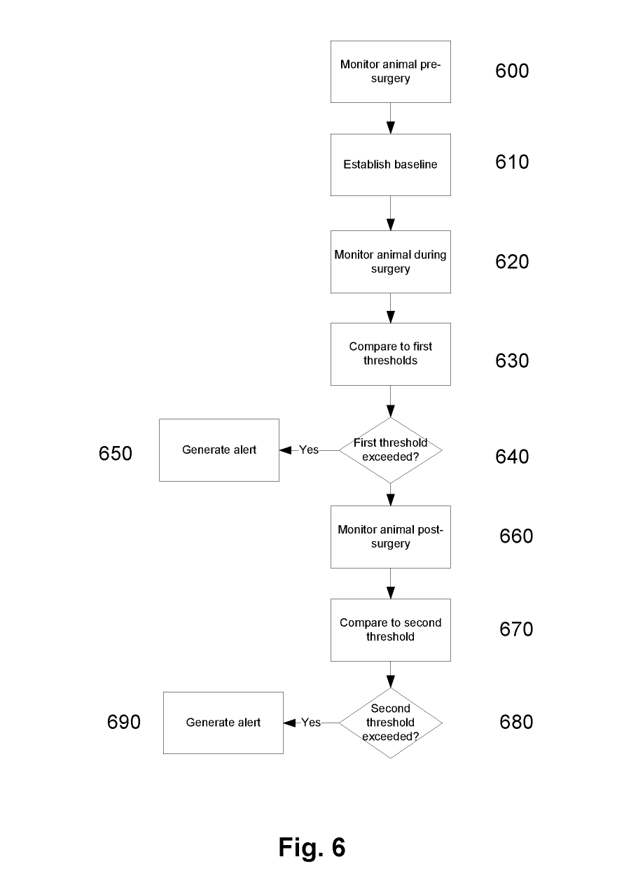

A fourth example of a method for use in monitoring an animal is shown in FIG. 6, and in particular for using in monitoring the animal before, during and after undergoing surgery/treatment.

At step 600 the animal is monitored using any of the above-described arrangements. At step 610 a baseline, which corresponds to predetermined first and second signals, is established. This may be achieved in any suitable manner, for example, manually or automatically, and by selecting instantaneous signals, or averaging a sample of signals over time, where signals correspond to first and second signals as described above.

At step 620, the animal is monitored during surgery/treatment. As discussed above, this may involve optionally connecting wires from one or more of the sensors or the base station 420, to existing/legacy monitors. At step 630, signals received by a base station are compared, periodically or more typically continuously, to the baselines established at step 610, and in turn the results of this comparison are compared to one or more first thresholds. At step 640 if the first threshold is exceeded an alert is generated at 650, which may include a visual indication on a display and/or an audible alarm.

The animal is also monitored after surgery, at step 660, for example during post-operative recovery and/or in the medium- to long-term. During this time, the signals received by the base station are compared, continuously or more typically periodically, to the established baseline, and the results of this comparison are in turn compared to one or more second thresholds at step 670.

In particular, the first and second thresholds are typically different. For example, in the event the first and second signals correspond to ECG and pulse-oximetry signals, during surgery when the animal is under general anaesthetic the heart beat, for example, is typically substantially lower than when the animal is ambulatory. Thus, the first threshold in respect of a heart beat will typically be indicative of a lower expected heart rate than the second threshold.

In any event, at step 680 if the second threshold is exceeded, an alert will be generated at step 690. In this regard, the alert generated at step 690 may be similar or different to the alert generated at step 650. For example, this alert may be generated remotely in the event of longer term monitoring, via email, SMS, or the like, however this is not essential.

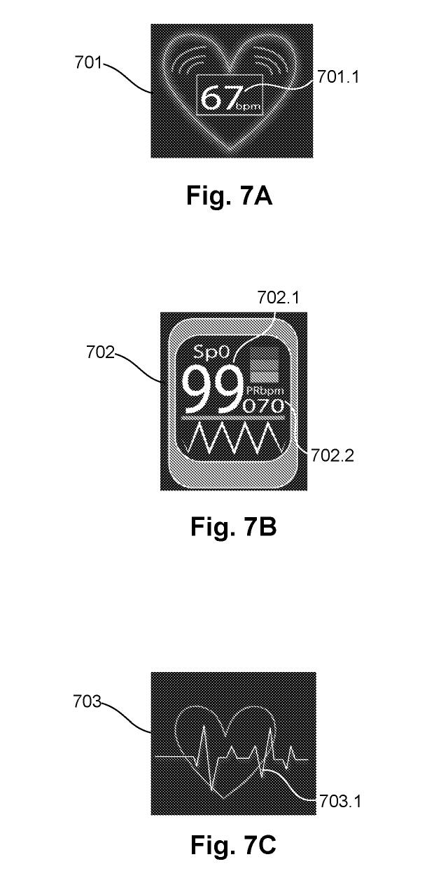

Examples of representations indicative of one or more indicators are shown in FIGS. 7A, 7B, and 7C.

FIG. 7A shows an example of a representation 701 indicative of a heart beat of an animal, including a numerical indicator of the number of beats per minute 701.1. FIG. 7B shows a further example of a representation 702 indicative of a heart beat and oxygen saturation of an animal, including a numerical indicator of oxygen saturation level 702.1, and a numerical indicator of the pulse rate of the animal 702.2. FIG. 7C shows a further example of a representation 703 indicative of electrical activity of the heart of an animal, including a graphical representation of the ECG 703.1. Thus, the representation may include graphical, numerical or symbolic representations of one or more of the signals monitored in respect of one or more than one animal.

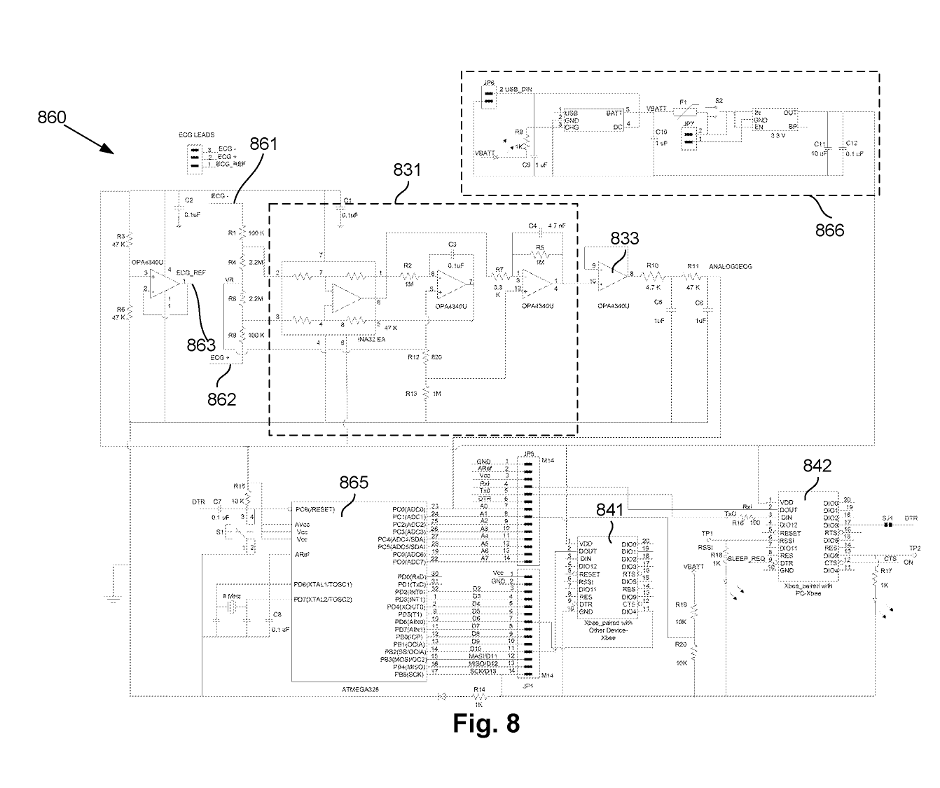

A further example of a collar for use in monitoring an animal is shown in FIG. 8. Features similar to those of the example described above have been assigned correspondingly similar reference numerals.

In this example, the collar 860 is intended for use with one or more second sensors worn on a non-neck region of the animal, such as a tail sensor for sensing pulse oximetry signals, and a base station, as described above.

In particular, the collar 860 includes three ECG electrodes 861, 862, 863 for sensing electrical activity of the animal's heart. The ECG signals obtained from the electrodes 861, 862, 863 are then filtered and amplified using an amplification/filtering circuit 831, and buffered using a unity gain amplifier 833. The resultant filtered ECG signals are then input to a microcontroller 865 which includes an ADC for digitising the filtered ECG signals. The microcontroller 865 may also perform additional signal processing on, and/or store, the ECG signals as described above.

The collar 860 further includes an XBee® transceiver 841 for receiving pulse oximetry signals from the tail piece, and an XBee® transceiver 842 for transmitting the ECG and pulse oximetry signals to the base station. In this regard, the microcontroller 865 is in electronic communication with the transceivers 841, 842 to control the transmission of signals, and to process any signals received. It will be appreciated that the transceivers 841, 842 may also transmit signals to the tail piece as well as receive signals from the base station, respectively, such as signals indicative of configuration data.

The collar 860 further includes a power supply circuit 866, which in this example includes a rechargeable battery. However, it will be appreciated that any suitable power source could be included, such as a non-rechargeable battery, or the like.

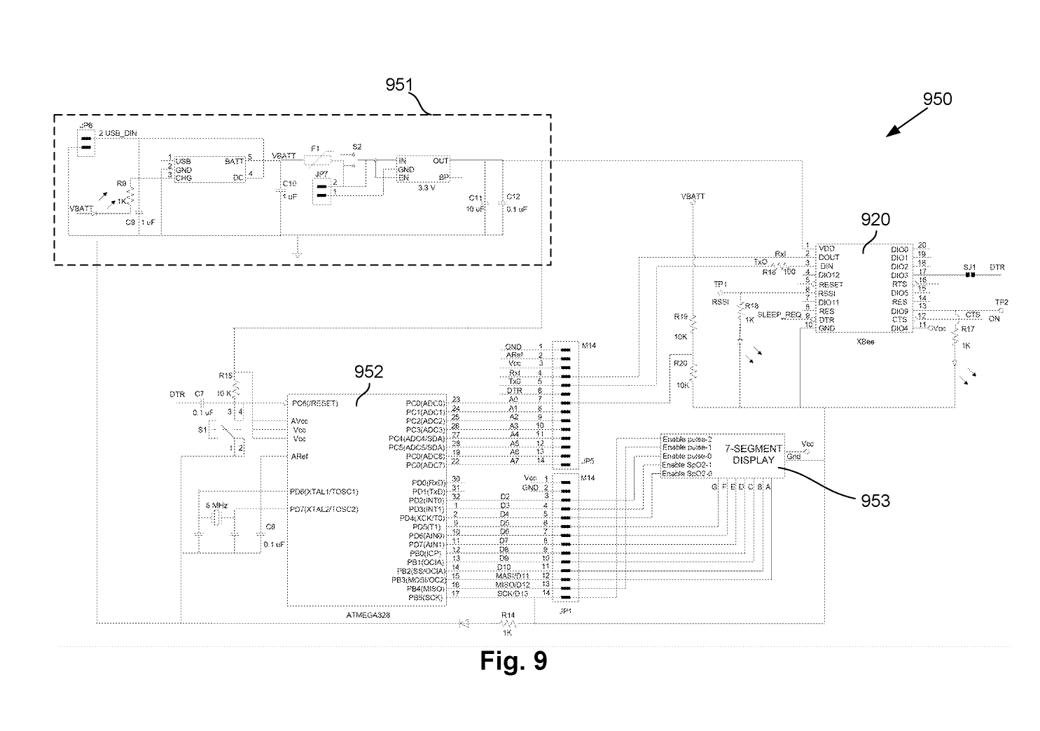

A further example of a tail piece for use in monitoring an animal is shown in FIG. 9. Features similar to those of the example described above have been assigned correspondingly similar reference numerals.

In this example, the tail piece 950 is intended for use with a first sensor worn on a neck region of the animal, such as in any of the examples described above, and in one example is for use with the collar of FIG. 8.

In particular, the tail piece 950 includes a pulse oximetry sensor for sensing the oxygen saturation of an animal. The pulse oximetry signals obtained from the sensor are then amplified, filtered and optionally buffered. The resultant filtered pulse oximetry signals are then input to a microcontroller 952 which includes an ADC for digitising the filtered pulse oximetry signals. The microcontroller 952 may also perform additional signal processing on, and/or store, the pulse oximetry signals as described in the examples above.

The tail piece 950 further includes an XBee® transceiver 920 for transmitting the pulse oximetry signals from the microcontroller 952 to the collar. It will be appreciated that the transceiver 920 may also receive signals from the collar, such as signals indicative of configuration data.

In addition, the tail piece 950 includes a display 953. In this regard, the microcontroller 952 is connected to the display 953, and thus may display any suitable indicator or representation thereof on the display 953. In one example, a representation of the battery status and connectivity, namely a pairing with collar, or the like may be displayed on the display 953, or representations indicative pulse oximetry signals, heart beat, an alarm or alert or any other indicators discussed above, or other configuration data.

The tail piece 950 further includes a power supply circuit 951, which in this example includes a rechargeable battery. However, it will be appreciated that any suitable power source could be included, such as a non-rechargeable battery, or the like.

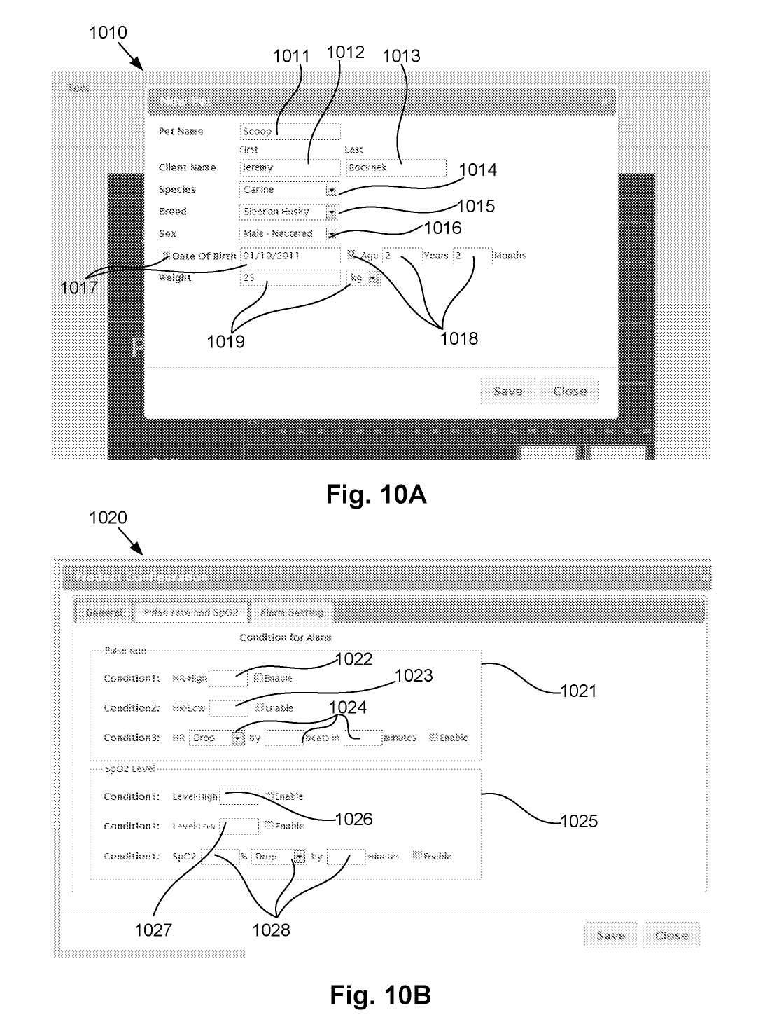

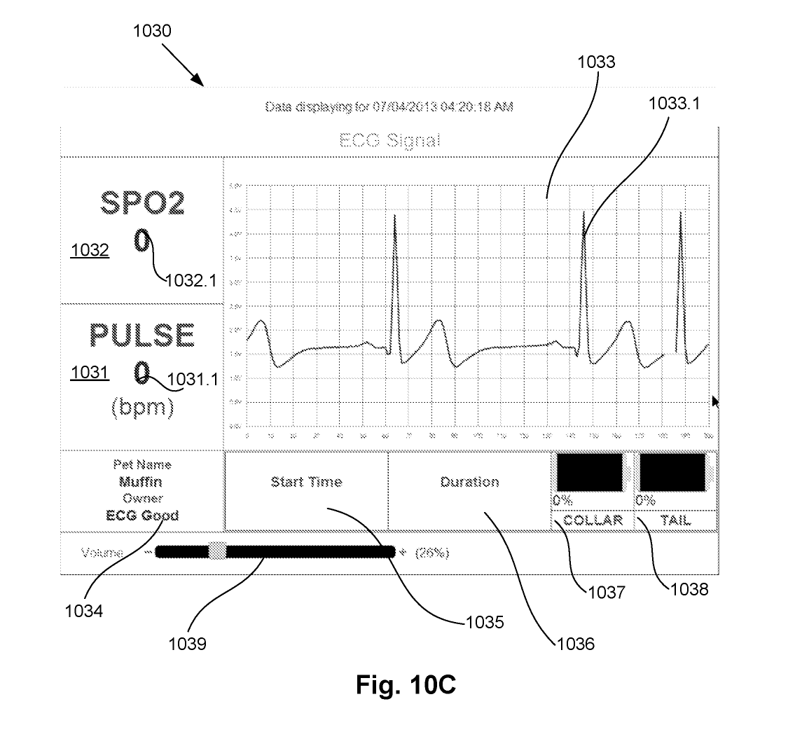

Examples of user interfaces displayed by an apparatus for use in monitoring an animal are shown in FIGS. 10A, 10B, and 10C. In this regard, as described above the computer application may be executed on a base station, a remote server, or as a cloud-based application, or the like, as discussed above.

In FIG. 10A, the user interface 1010 shows one example of a form for capturing details about an animal. In particular, the form allows the user to input the animal's name 1011, also referred to as the pet's name, and the client's first name 1012 and last name 1013, also referred to as the owner's name. In addition, the user may input the animal's species 1014, breed 1015, and sex 1016, including whether the animal has been previously desexed. Furthermore, the user has the option to input the animal's date of birth 1017 and/or its age 1018, and the animal's weight 1019. In this regard, the animal's details may be used to aid in identifying stored representations, such as naming files, file meta-data, and the like. Additionally or alternatively, at least some of the animal's details may be used to select/display reference values, such as the animal's previous indicators, or reference indicators typical of the species and/or breed and/or sex, and the like. In a further example, the reference values may be used to configure alarms, for example, in the event animal's heart rate falls outside of a reference heart rate determined based upon the animal's breed.

FIG. 10B shows an example of a user interface 1020 that allows the user to configure one or more alarms on the basis of one or more conditions. For example, the user may configure any one or more pulse rate alarms 1021 to indicate when a pulse rate of the animal is over a predefined threshold 1022, under a predefined threshold 1023, or changes at a predefined rate 1024. In respect of oxygen saturation, the user may configure one or more alarms 1025 to indicate that the oxygen saturation exceeds 1026, or does not meet 1027, predefined levels, or when there is a predefined change in oxygen saturation 1028. However, it will be appreciated that alarms may be configured on the basis of any other biological attribute, and in some examples the alarms may be configured automatically on the basis of the breed and/or species and/or conditions of the animal, such as described above.

The example user interface 1030 shown in FIG. 10C includes a number of representations, such as a pulse oximetry representation 1032 indicative of oxygen saturation 1032.1, a heart rate representation 1031 indicative of heart beat 1031.1, and an ECG representation 1033 indicative of electrical activity of the heart 1033.1. In addition, the user interface 1030 displays the animal's name, and the owner's name 1034, and a start time 1035 and duration 1036 during which the displayed data was sensed. Furthermore, the user interface 1030 displays the remaining battery life of the collar 1037 and tail piece 1038. However, it will be appreciated that in other examples, further representations of indicators and/or configuration data may be displayed, such as location of the animal, alerts/alarms, and the like.

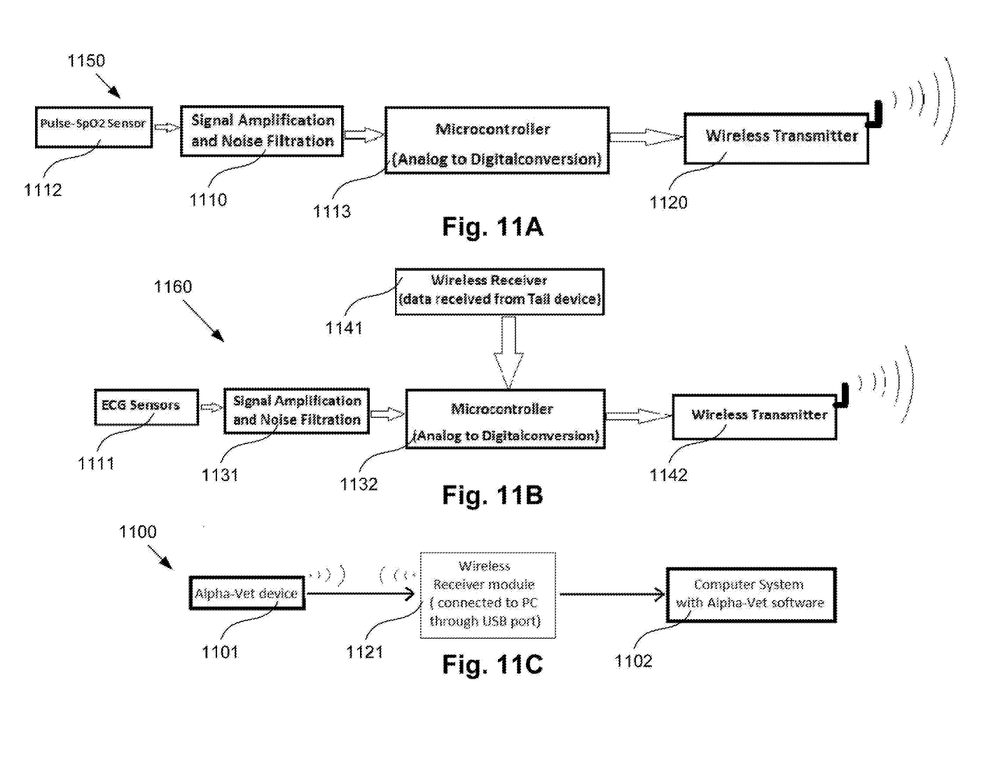

A further example of a tail piece, collar, and apparatus for use in monitoring an animal is shown in FIGS. 11A, 11B, and 11C, respectively. Features similar to those of the example described above have been assigned correspondingly similar reference numerals.

In this example, the tail piece 1150 shown in FIG. 11A includes a pulse oximetry sensor 1112 for using in monitoring an animal's oxygen saturation and pulse, and signals from the sensor 1112 are amplified and filtered to remove noise 1110. A microcontroller 1113 digitises the filtered signals, for example, using an ADC and transmits the resultant digitised signals to the collar using a wireless transmitter 1120.

In one particular example, the tail piece 1150 has the following performance characteristics.

Physical Characteristics
87 mm×64 mm×28 mm (W×L×H)
140 gm
Pulse and SpO2 sensor

Wireless Module
Data Rate: 250 Kbps
Module Interface: UART
Supply Voltage Range: 2.1V to 3.6V
Kit Features: XBee Pro Platform, 315 m Outdoor RF Line-of-sight Range, 250 Kbps Data Rate, Chip Antenna
Tool/Board Application: Mesh Networking
Tool/Board Applications: Wireless Connectivity
Type: RF Module

Battery
3.7 volts
1000 mAH
Li—Po battery
Charging time—6 hours

Charger
AC-DC adapter
100-250 V AC 50-60 Hz Input
5 V DC Output

In FIG. 11B, the collar 1160 includes ECG sensors 1111 which sense electrical activity of the animal's heart. The ECG signals are then amplified and filtered to remove noise 1131. The filtered signals are then digitised by a microcontroller 1132, which includes an ADC function. The microcontroller 1132 also determines signals from a wireless receiver 1141, which are typically received from the tail piece 1150 in the form of pulse oximetry and pulse signals. The wireless transmitter 1142 transmits the ECG signals and/or pulse oximetry and pulse signals to the base station.

In one particular example, the collar 1160 has the following performance characteristics.

Physical Characteristics:
87 mm×64 mm×28 mm (W×L×H)
165 gm
ECG connectors with cables
ECG Acquisition
3 leads, simultaneous.
Input impedance>100 MegaOhm
Frequency response 0.05-150 Hz-3 dB
Sensitivity: 5, 10, 20 mm/mV+/−10%
Dynamic range: +/−10 mV
ADC resolution: 10 bits
Acceptable electrode offset: +/−300 mV per AAMI and EC-11 specifications.
A/D 1000 samples/sec.

Wireless Module:
Data Rate: 250 Kbps
Module Interface: UART
Supply Voltage Range: 2.1V to 3.6V
Kit Features: XBee Pro Platform, 315 m Outdoor RF Line-of-sight Range, 250 Kbps Data Rate, Chip Antenna
Tool/Board Application: Mesh Networking
Tool/Board Applications: Wireless Connectivity
Type: RF Module

Battery:
3.7 volts
1000 mAH
Li—Po battery
Charging time—6 hours

Charger:
AC-DC adapter
100-250 V AC 50-60 Hz Input
5 V DC Output

FIG. 11C shows a further example of an apparatus 1100 for use in monitoring an animal, the apparatus including a collar and tail piece shown generally at 1101, and a base station 1102. In this example, the base station 1102 is coupled to a wireless receiver 1121 which is connected to the base station 1102 via a cable, such as to a universal serial bus (USB) port on the base station 1102. However, this is not essential, and in other examples the wireless receiver 1121 may be provided in the base station 1102, or connected to the base station 1102 in any suitable manner.

In one particular example, the apparatus 1100 includes the following environmental operating limits.
Operating: 59 to 95° F. (15 to 35° C.); 30 to 75% humidity (non-condensing); 760 mm Hg+/−20%.;
Storage/Shipping: 4 to 120° F. (−15 to 50° C.); 30 to 95% humidity (noncondensing); 760 mm Hg+/−20%

In a further example, the base station 1102 includes the following minimum requirements.
OS: Window XP (32-bit or 64-bit), Window Vista (32-bit or 64-bit) and Window 7 (32-bit or 64-bit)
Storage: Minimum 8 GB
Processor: Minimum a Pentium 2 266 MHz processor
RAM: 512 MB or higher for Windows XP, 1 GB or higher for Windows Vista, 2 GB or higher for Windows 7

Thus, the functions of this example, for example amplification, filtering, digitisation, transmission, and the like, may be performed in any suitable manner, including as outlined in any of the examples described above. Furthermore, the performance characteristics are provided for example only, and it will be appreciated that the apparatus 1100 may include any suitable performance characteristics.

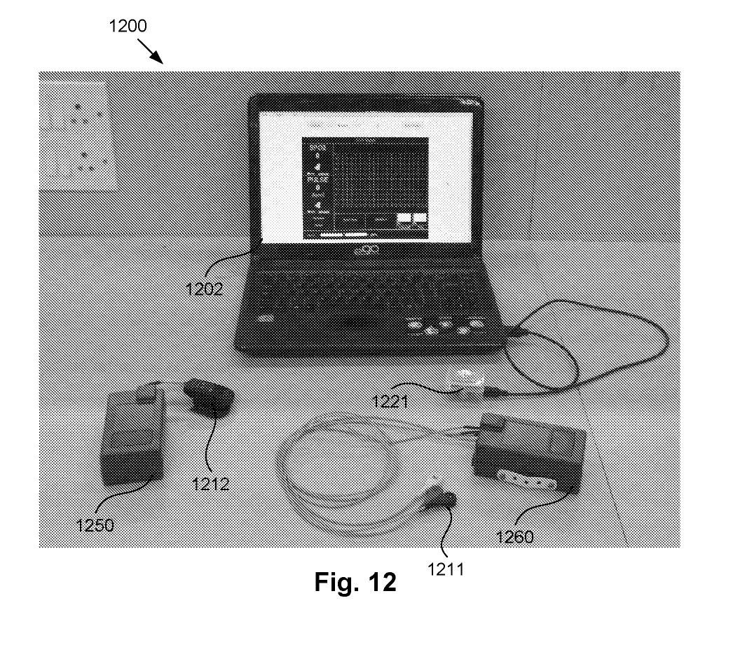

A further example of an apparatus for use in monitoring an animal is shown in FIG. 12. Features similar to those of the example described above have been assigned correspondingly similar reference numerals.

In this example, the apparatus 1200 includes a tail piece 1250 including pulse oximetry sensor 1212, and a collar 1260 including ECG sensors 1211. A base station 1202 is coupled to a wireless receiver 1221 for receiving signals from the collar 1260. The base station 1202 also displays a user interface. The apparatus 1200 may function similarly to any one of the above examples.

As will be appreciated from the above, the apparatus of any of the above examples may be used in numerous applications. For example, animals of any type may be monitored pre-operatively following premedication administration. Currently, animals after premedication are very excited and agitated, which precludes use of existing monitoring equipment that the animal can simply strip off, remove or damage. Therefore existing solutions do not allow for monitoring animals following premeditation. In contrast, the apparatus allows a user to monitor the animal for either the effectiveness of the premedication of for possible adverse reactions using wearable and wireless sensors, which the animal cannot remove or otherwise damage.

Additionally, the apparatus may be used for post-operative monitoring of animals post-surgery, and in one example, where the animal is conscious and/or ambulatory. During the recovery phase of anaesthesia, animals may be disoriented and thrash about, and in some cases can be dangerous to handle. Current monitoring equipment is strongly affected by movement and is often torn off or otherwise removed by the animal. The apparatus however, enables monitoring of a moving and/or ambulatory animal as the apparatus is wireless and the sensors are wearable and hence may provide monitoring of animals post-operatively to ensure recovery progresses as expected and to detect any issues early.

Furthermore, post-operative monitoring of animals where the animal may pose a danger to the handler could be conducted using the apparatus. In this regard, typically wild animals, zoo animals, and most domestic farm animals pose a serious risk to their human handlers. Current equipment typically requires direct contact with the animal in order to place, adjust and remove the equipment. In contrast, the apparatus may allow handlers to monitor the animals from a safe distance or behind protective enclosures, and thus avoid injury or harm.

Similarly, pre-operative monitoring of dangerous animals following premedication may also be performed using the apparatus. Wild animals are unpredictable at the best of times, and the effect of medication on these animals is also often unpredictable and in some situations may make the animal more dangerous. However, the apparatus allows for such dangerous animals to be safely monitored pre-operatively. For example, a lion premedicated with a pole syringe and wearing the apparatus, may then be safely monitored for adverse or normal reactions to the medication at a safe distance.

In situations where post-operative monitoring is required on animals that, if restrained, would suffer undue stress, the apparatus also provides significant advantages. In this regard, many of the animals that require surgical intervention are flight/prey animals, and in situations of stress where medical attention is required, restraining these animals will only increase stress, due to the inability of the animal to move/flee. Accordingly, this may further compound the disease state of the animal. The apparatus may therefore allow these animals to move while being monitored, thereby reducing their stress and speeding their recovery.

The apparatus may also be used to monitor ill animals in a hospital setting where the animal's movement would prevent monitoring by existing devices. Unlike humans, animals as a group do not sit still and do not tolerate external irritants such as clips and wires. Despite the use of cones, bandages and drugs these animals will actively remove and or destroy such items. The apparatus described herein is wireless and typically does not include clips or the like, thereby removing the irritants and allowing the animal to accept monitoring when currently none can be done.

Additionally, the apparatus may be used in monitoring ill animals in a farm setting. Farmers lead exceptionally busy lives and typically do not have a lot of time to devote to any one animal. In addition, veterinary visits can be expensive. Therefore, the apparatus allows an individual animal to be monitored from a central location, such as in the home, from a veterinary practice, centrally, or the like, for example, up to 24 hrs per day and 7 days per week, if desired or necessary. Indicators or representations thereof or the like may be forwarded and even monitored by the veterinarian, for example, in real time, and decisions can be made for a fraction of the cost. Problems may be identified before they become catastrophes and animal lives and costs can be saved.

The apparatus may also be used in monitoring pregnant animals in a farm setting. Birthing time is very stressful and time consuming for farmers. The apparatus may be configured with alarms, for example as discussed above, to warn the farmer when drops in temperature, increases in heart rate, or any other suitable indicator, indicates that the birth is imminent, or that the pregnant animal requires medical attention. This may free up considerable amounts of time for the farmer and at the same time increase the ability to monitor these animals, thereby reducing mortality and cost with birthing problems.

Use in monitoring of pregnant animals may not be limited to the farm setting, and in other applications the apparatus may be used to monitor pregnant animals in other settings, such as the home or a commercial setting. For example, animal breeders of all types, not just commercial farmers would benefit from enhanced monitoring ability during birthing times. Many times animals give birth at night and no one is available to assist, which may be very traumatic for the family, handler or individual, dangerous for the animals and can be very expensive if veterinary care is required or infant mortality is high. Also, current monitoring devices have clips and wires which can cause increased stress on the mother and result in her delaying the birth thereby resulting in birthing complications. Therefore wearable sensors and wireless transmission, allow the apparatus to be worn by the animal during pregnancy and birth, without causing the animal undue stress, and allow for the early detection and intervention of any health problems.

When any new animal is brought to a new farm, or other new environment, it is a highly stressful event which increases the animal's susceptibility to illness. Excessive handling in this situation may only further increase an animal's stress. The apparatus includes the advantage of creating a stress free mechanism for closely monitoring these valuable animals, through wireless monitoring.

Additionally, the apparatus may be used for monitoring any type of ill animal in a zoo setting. Zoo animals are both rare and valuable, however they are also dangerous and wild. Over handing of the animals may cause undue stress to these animals placing them and their handlers in danger. The apparatus provides a hands-free remote monitoring system where none currently exist.

However, use of the apparatus in a zoo setting is not limited to ill animals, and the apparatus may also be used to monitor animals under stress. New animals introduced to the zoo are under as much or more stress and pressure then newly introduced livestock. They may be extremely rare and valuable. Being able to closely monitor them at a distance is an invaluable tool and currently none exist. This information, such as indicators determined using the apparatus, could also be shared with other zoos around the world to assist them in introducing new animals into their habitats.

The apparatus may also be used in monitoring pregnant animals in a zoo setting. Breeding programs are an integral part of a zoo's mandate, a task which is exceptionally difficult. Pregnant females typically avoid contact, are highly susceptible to stress induced abortions, and can also pose an extreme risk to both themselves and their handlers. The apparatus therefore at least partially ameliorates the current monitoring dilemmas, by providing wireless and centralised monitoring.

Furthermore, the apparatus may be used in monitoring animals of any type during transport. In this regard, during transport animals are susceptible to stress which may affect their health, performance, or the like. Therefore, the ability to remotely monitor animals during transportation allows users to ensure that the animal is not unduly stressed, pre-empt any effects of stress on the animal's health, and potentially tailor the animals activities pre and post-transportation, such as curing reintroduction into an environment, or influencing a training or racing schedule, or the like. For example, this could be used to monitor breathing during transport such as flights, which is particularly important for animals that are brachycephalic, such as bull dogs, pugs, or the like.

In some examples, it may be desirable for the apparatus of any one of the above examples to be used in logging data. In this regard, data collected using the apparatus may be centrally logged, for example, by a proprietor or distributor of the apparatus, a veterinary practice, a farmer, a head zookeeper, or the like. In this respect, the data may provide indicators of previous, current, and potentially the future health of the animal.

In one example, central or remote logging is conducted by the proprietor or distributor of the apparatus, or another centralised agency. In this regard, logging of data received from a plurality of animals may be conducted over extended periods of time, for example continuously or periodically during the entire lifetime of an animal. Information collected in this manner may provide valuable insights into the health of an animal or offspring over the course of its life, and in particular, during stressful times such as birth, introduction into a new environment, pregnancy, transportation, and the like. In some examples, the data logged in respect of an individual animal may be complied into a health “passport” for the animal, which may be provided to prospective buyers, investors, new owners, or the like.

It will be appreciated that data may be logged according to any suitable method, for example over a network, such as the Internet, using proprietary software, cloud computing, one or more remote servers, or the like. In one example, the logging is conducted using a remote processing system such as discussed above in respect of FIG. 4. In this regard, the centralised logging may be performed using the base station, or alternatively may be used in addition to base stations, for example by centrally logging data from one or more base stations. Furthermore, the data received from one or more apparatus for monitoring animals may be centrally stored, encrypted, further processed, or the like.

In addition, the centralised agency may provide reports on individual animals, or groups of animals, according to a charging model. This may involve consumers purchasing a subscription which provides temporal or usage restricted access to reports on one or more animals. Additionally or alternatively, costs may be charged on a per report basis, or according to a cost per health “passport”.

In a further example, an apparatus according to any one of the above examples may be used to monitor one or more animals during selected periods, for example, during periods of time where the animal is susceptible to experiencing high levels of stress.

For example, the apparatus may be used to monitor an animal during transportation. Not only does this allow the animals stress levels to be monitored in real-time, but the data determined during this period may be logged and stored either centrally or using the base station. In this regard, the stored data may be used, for example in respect of performance animals, to motivate a change in the animal's training, or to indicate that the animal needs to be withdrawn from a race. Furthermore, the stored data may be used to determine optimal transportation techniques for minimising health impacts on animals, or the like.

In another example, the apparatus may be used to monitor an animal during pregnancy. In this regard, the experiences of a mother during pregnancy and labour may have long-term effects on the offspring. For example, if a mare undergoes a period of extreme stress during labour, her foal may experience long term health problems as a result. Therefore, data logged and stored during pregnancy and birth may provide valuable information on the potential future health, performance, and the like of the offspring. In this regard, such information may be used to supplement genetic testing, genealogy, and the like and may be of interest to potential buyers, investors, trainers, and the like.

The abovementioned method and apparatus for monitoring animals provides numerous advantages, such as allowing long term monitoring of ambulatory animals without constraining movement.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described. Thus, for example, it will be appreciated that features from different examples above may be used interchangeably where appropriate.

1) A method for monitoring an animal, the method including an electronic processing device for:
a) wirelessly receiving at least one signal indicative of first and second biological attributes of the animal, wherein:
i) the at least one first biological attribute is at least partially sensed using at least one first sensor worn on a neck region of the animal; and,
ii) the at least one second biological attribute is at least partially sensed using at least one second sensor worn on a non-neck region of the animal; and,
b) generating at least one indicator at least partially indicative of the first and second biological attributes.

2) A method according to claim 1, wherein the method includes displaying a representation of the at least one indicator.

3) A method according to claim 1, wherein the method includes:
a) generating the at least one signal from second signals received from the second sensor using a receiver provided in proximity to the first sensor and from first signals from the first sensor; and,
b) transmitting the at least one signal from a transmitter provided in proximity to the first sensor.

4) A method according to claim 1, wherein the method includes, in the electronic processing device:
a) determining attribute values indicative of the first and second biological attributes; and,
b) determining the indicator using the attribute values.

5) A method according to claim 4, wherein the method includes, in the electronic processing device:
a) comparing the attribute values to at least one reference; and,
b) generating the indicator using a result of the comparison.

6) A method according to claim 5, wherein the reference is at least one of:
a) derived from a normal population;
b) a predetermined threshold;
c) determined from predetermined values; and,
d) indicative of previously determined attribute values.

7) A method according to claim 6, wherein the previously determined attribute values are determined prior to the animal undergoing at least one of:
a) surgery; and,
b) treatment.

8) A method according to claim 1, wherein the animal is a non-human animal.

9) A method according to claim 1, wherein the animal is ambulatory.

10) A method according to claim 1, wherein the non-neck region includes any one of:
a) a tail; and,
b) a hind leg.

11) A method according to claim 1, wherein the at least one second biological attribute includes at least one of:
a) a heart rate; and,
b) an oxygen saturation.

12) A method according to claim 1, wherein the at least one first biological attribute includes at least one of:
a) electrical activity of a heart of the animal; and,
b) a respiration rate.

13) A method according to claim 12, wherein the second signal is indicative of an electrocardiograph or electrocardiogram.

14) A method according to claim 1, wherein the first or second sensor includes at least one of:
a) a photodetector;
b) an induction sensor;
c) an elastomeric sensor;
d) a pressure sensor;
e) a current sensor;
f) a voltage sensor;
g) an impedance sensor;
h) a resistance sensor; and,
i) an accelerometer.

15) An apparatus for use in monitoring an animal, the apparatus including:
a) at least one first sensor worn on a neck region of the animal for at least partially sensing at least one first biological attribute of the animal;
b) at least one second sensor worn on a non-neck region of the animal for at least partially sensing at least one second biological attribute of the animal; and,
c) a base station including an electronic processing device for:
i) wirelessly receiving at least one signal indicative of the first and second biological attributes; and,
ii) generating at least one indicator at least partially indicative of the first and second biological attributes.

16) An apparatus according to claim 15, wherein the apparatus includes a first and second wearable support being worn by the animal, the first and second wearable supports supporting the first and second sensors respectively.

17) An apparatus according to claim 16, wherein the first wearable support includes a wireless transmitter for transmitting the at least one signal.

18) An apparatus according to claim 15, wherein the first or second sensor includes at least one of:
a) a photodetector;
b) an induction sensor;
c) an elastomeric sensor;
d) a pressure sensor;
e) a current sensor;
f) a voltage sensor;
g) an impedance sensor;
h) a resistance sensor; and,
i) an accelerometer.

19) An apparatus for use in monitoring an animal, the apparatus including:
a) a first wearable support being worn at least partially on a neck region of the animal, the first wearable support including:
i) at least one first sensor for at least partially sensing at least one first biological attribute of the animal and generating a first signal indicative of the at least one first biological attribute;
ii) a first wireless receiver for receiving at least one second signal indicative of at least one second biological attribute of the animal; and,
iii) a first wireless transmitter for transmitting at least one signal indicative of the first and second biological attributes using the first and second signals; and,
b) a second wearable support being worn on a non-neck region of the animal, the second wearable support including:
i) at least one second sensor for at least partially sensing the second biological attribute and generating the second signal; and,
ii) a second wireless transmitter for transmitting the second signal.

20) An apparatus that communicates with a collar for monitoring an animal, the apparatus including:
a) a wearable support including at least one sensor for at least partially sensing at least one biological attribute of the animal, the wearable support being worn at least partially on a non-neck region of the animal; and,
b) a wireless transmitter provided on the wearable support for transmitting at least one signal indicative of the biological attribute to the collar.

21) A collar for use in monitoring an animal, the collar being a wearable support worn at least partially on a neck region of the animal, the collar including:
i) at least one sensor for at least partially sensing at least one first biological attribute of the animal and generating a first signal indicative of the at least one first biological attribute;
ii) a first wireless receiver for receiving at least one second signal indicative of at least one second biological attribute of the animal; and,
iii) a first wireless transmitter for transmitting at least one signal indicative of the first and second biological attributes using the first and second signals.
